(19) United States
(12) Patent Application Publication
(10) Pub. No.: US 2001/0006677 A1
(43) Pub. Date: Jul. 5, 2001

(54) EFFERVESCENCE POLYMERIC FILM DRUG DELIVERY SYSTEM

(76) Inventors: JAMES W. MCGINITY, AUSTIN, TX (US); JOSEPH R. ROBINSON, WISCONSIN, WI (US)

Correspondence Address: MERCHANT & GOULD, P O BOX 2903, MINNEAPOLIS, MN, 55402-0903, US

(21) Appl. No.: 08/741,432

(22) Filed: Oct. 29, 1996

(51) Int. Cl. A61K 9/70; A61K 9/46

(52) U.S. Cl. 424/449; 424/435; 424/466

(57) ABSTRACT

According to the present invention, effervescent controlled release water soluble or swellable hot-melt extruded films are provided. Such films comprise a hot-melt extrudable water soluble or swellable binder, an active ingredient, an effervescent couple and optionally another compound such as a plasticizer. The films are made by a hot-melt extrusion process. Bioadhesive effervescent films can also be made by the invention.

### FIELD OF THE INVENTION

This invention relates to an effervescent composition and a method of preparing same. More specifically, it relates to a hot-melt extruded effervescent film having a controlled rate of disintegration.

### BACKGROUND OF THE INVENTION

Effervescent granules have found a variety of uses over the years. These include their use in dental compositions containing enzymes, contact lens cleaners, washing powder compositions, beverage sweetening tablets, chewable dentifrices, denture cleaners, surgical instrument sterilizers, effervescent candies, as well as in many pharmaceutical formulations. Pharmaceutical formulations that include effervescent granules, by way of example, are formulations of analgesics, antibiotics, ergotamines, digoxin, methadone and L-dopa.

Polymer film-coated effervescent granules have been described. In particular, polymers such as cellulose acetate phthalate or hydroxypropyl methylcellulose have been used. Such coatings have been introduced in order to increase tablet stability as well as to control dissolution rate and to target particular regions of the gastrointestinal tract.

Schiraldi et al. (U.S. Pat. No. Re. 33,093) relates to a bioadhesive controlled-release medicament containing an extruded single or multi-layered film. These films comprise hydroxypropyl cellulose, a homopolymer of ethylene oxide, a plasticizer and optionally a water insoluble polymer. The films were made by an extrusion process.

Harwood et al. (Drug. Develop. Indust. Pharm. (1982), 8(5), 663-682) relates to compression molded films of various pilocarpine salt/hydroxypropyl cellulose compositions.

Machida et al. (Drug. Des. Delivery (1989), 4(2), 155-61) relates to the preparation of intragastric buoyant pharmaceutical preparations which, in one embodiment, comprise a drug-containing film, an effervescing film containing NaHCO₃ and an outer drug release regulating film.

U.S. Pat. No. 4,615,697 to Robinson relates to a buccal delivery system, combined with bioadhesive materials to keep the system in place in the oral cavity for an extended period of time. The treating agents are described as released in a controlled manner for both local and systemic effects.

Drug-dispensing films are disclosed in U.S. Pat. No. 3,641,237. The films are prepared by polymerization of lower alkoxy, lower alkyl acrylates and methacrylates along with 0-40 percent of a hydrophilic acrylic monomer in the presence of a cross-linking agent. Various monomers are disclosed, such as: hydroxyalkyl acrylates and methacrylates, salts of α,β-unsaturated organic acids and strong acid salts of polymerizable ethylenically unsaturated amine-containing monomers.

Several materials, which in the presence of water adhere to the mucus membrane, have been used alone or in combination with one or more active agents to treat various pathological conditions. Examples of such materials are the complex of sulfated sucrose and aluminum hydroxide, known as sucralfate, and are available under the name of Carafate® (Marion Laboratories, Inc., Kansas City, Mo.). Sucralfate is used alone or in conjunction with an antacid to treat duodenal ulcers. Another adherent material, designed for used in the buccal cavity, is a combination of gelatin, pectin, and sodium carboxymethylcellulose in a plasticized hydrocarbon gel available under the name of Orabase® (Hoyt Laboratories Division of Colgate-Palmolive Co., Needham, Mass.). A mucosal adherent ointment based upon partly neutralized polymethacrylate acid methyl ester was reported by Bremecher et al. Arzneim-Forsch/Drug Res., 33,591(1983). That ointment was reported to show a pseudoplastic quality without any thixotropic effect, good mucosal adhesion and no local irritation.

U.S. Pat. No. 4,226,848 relates to a composition for adhering a pharmaceutical preparation to the mucosa of the oral or nasal cavities. The composition disclosed contains a water-swellable and mucosa-adherent polymeric matrix comprising (a) about 50 to about 95 percent by weight of a cellulose ether and (b) about 50 to about 5 percent by weight of a homo- or copolymer of acrylic acid or a pharmaceutically acceptable salt thereof, with a pharmaceutically effective amount of a medicament dispersed therein.

U.S. Pat. No. 4,615,697 relates to a composition which includes a bioadhesive and an effective amount of a treating agent. The bioadhesive comprises a water-swellable, but water-insoluble, fibrous, cross-linked carboxy-functional polymer. The polymer contains (a) a plurality of repeating units of which at least about 80 percent contain at least one carboxyl functionality, and (b) about 0.05 to about 1.5 percent cross-linking agent substantially free from polyalkenyl polyether, said percentages being based upon the weights of unpolymerized repeating units and cross-linking agent, respectively. The polymer is available under the name of Noveon AA-1® (B.F. Goodrich Chemical Company).

Lindberg (Acta. Pharm. Suec. (1988), 25, 239-246) relates to a continuous wet granulation method for preparing effervescent granules. The process described includes the steps: (1) mixing powdered citric acid and NaHCO₃ in the hopper of a Baker Perkins cooker extruder and granulating the mixture with ethanol.

U.S. Pat No. 5,178,878 to Wehling et al relates to an effervescent dosage form incorporating microparticles which are susceptible to rupture upon chewing or which are adapted to provide substantially immediate release of the pharmaceutical ingredients contained in the microparticles. The microparticles comprise a drug encapsulated in a protective material. The microparticles are then mixed with an effervescent agent and then the mixture compressed into tablets.

Kond et al., in U.S. Pat. No. 5,223,246, relates to a water soluble effervescent composition prepared by hot-melting (1) an active component and (2) an acid and a carbonate for effervescence, and (3) a water soluble adjuvant whose melting point is not lower than 40° C. The effervescent composition was prepared by mixing the active agent, the acid, the carbonate and the water soluble adjuvant and then heating the entire mixture to melt the adjuvant and subsequently cooling the mixture to room temperature while stirring to form effervescent particles.

Hot-melt extrusion processes in the art have generally required extremely elevated temperatures (>150° C.). These temperatures could degrade extruded materials such as those that combine to form an effervescent composition. A need continues to exist in the art for improved effervescent film preparations useful in a hot-melt extrusion process.

### SUMMARY OF THE INVENTION

In one aspect, the present invention provides improved effervescent film formulations preparable in a hot-melt extrusion process. In some embodiments, an effervescent water soluble or swellable controlled release hot-melt extruded single or multi-layered thin film is provided comprising: a water soluble or swellable hot-melt extrudable effervescent film binder present in an amount of about 40% to about 99.9% by weight; an effective amount of an active ingredient present in an amount of about 0.05% to about 60% by weight; a plasticizer present in an amount of about 0% to about 50%; and an effervescent couple present in an amount of about 0.1% to about 60%. In some embodiments, the weight percentages of these formulations are based upon the final weight of the effervescent film. In some embodiments, the formulations provide for a rapid rate of release of an active ingredient that ranges from immediate to a period of about 10 minutes. Other embodiments of the present invention provide a solid pharmaceutical dosage form adapted for direct oral or buccal administration, i.e., for direct insertion into the mouth of a patient.

Yet other embodiments of the invention provide an effervescent, bioadhesive, water soluble or swellable, controlled release hot-melt extruded single or multi-layered film for the rapid, controlled or sustained local or systemic delivery of an active ingredient. In some formulations, delivery of the active ingredient is provided by attachment of the film to oral cavity tissue. A release of the active ingredient may be tailored according to the dosage profile desired, and may span several hours. The effervescent bioadhesive formulation in some preparations may be created so as to be capable of adhering to the oral cavity mucosa, gingiva, buccal cavity, sublingual cavity and other locations of the mouth.

In another aspect, the present invention provides a method for improving the taste of a film formulation. It has been found that combination of the effervescent film with other ingredients can provide effective taste masking of particularly poor tasting compounds. This aspect of the invention thus provides a dosage form which offers both immediate release and effective taste masking.

The effervescent films of the invention may be used in pharmaceutical, veterinary, horticultural, household, food, culinary, pesticidal, agricultural, cosmetic, herbicidal, industrial, cleansing, confectionery and flavoring applications.

Formulations incorporating the effervescent film may further include one or more additional adjuvants and/or active ingredients which can be chosen from those known in the art including flavors, diluents, colors, binders, filler, surfactant, disintegrant, bioadhesive, penetration enhancer, protease inhibitor stabilizer, compaction vehicles, and non-effervescent disintegrants.

The effervescent film of the invention may include an effervescent couple in the form of an effervescent granule or granules. In some aspects, the effervescent granules included in the effervescent film do not include therapeutic compounds or other active ingredients.

The present invention also provides a method of preparing an effervescent rapid release hot-melt extruded water soluble or swellable film. In some embodiments, the method comprises: providing a hot-melt extruded effervescent granule comprising: a hot-melt extrudable effervescent granule binder and an effervescent couple; mixing said effervescent granule with a water-soluble or swellable hot-melt extrudable effervescent film binder, an effective amount of an active ingredient, and optionally a plasticizer to form an effervescent mixture; and hot-melt extruding said effervescent mixture to form said film.

The present invention also provides a method for preparing a bioadhesive effervescent controlled release hot-melt extruded soluble or swellable two-layered thin film comprising: providing a bioadhesive thin film; mixing an effervescent couple with a hot-melt extrudable water-soluble or swellable effervescent film binder, an effective amount of an active ingredient and optionally a plasticizer to form an effervescent mixture; hot-melt extruding said effervescent mixture to form an effervescent single-layered hot-melt extruded water soluble or swellable film; and pressing said effervescent single-layered hot-melt extruded water soluble or swellable film onto said bioadhesive thin film.

These films may be further described in some embodiments as having localized regions of high concentration of the effervescent couple dispersed within the hot-melt extrudable effervescent film binder.

The hot-melt extrusion process herein advantageously allows for extremely short exposure times of components to elevated temperatures as well as a higher throughput than batchwise hot-melt methods. Additionally, the process herein does not require the use of solvents as is required by prior art methods.

Effervescence can be defined as the evolution of bubbles of gas in a liquid. As set forth in chapter 6 of Pharmaceutical Dosage Forms: Tablets Volume I, Second Edition (A. Lieberman, ed., 1989, Marcel Dekker, Inc.; the entirety of which is hereby incorporated by reference), effervescent mixtures have been used medicinally. As discussed in this text, and as commonly employed, an effervescent tablet is dissolved in water to provide a carbonated or sparkling liquid drink.

Other features, advantages and embodiments of the invention will be apparent to those skilled in the art from the following description, examples and appended claims.

### BRIEF DESCRIPTION OF THE DRAWINGS

The following drawings are part of the present specification and are included to further illustrate certain aspects of the invention. The invention can be better understood by reference to one or more of the drawings in combination with the detailed description of the specific embodiments presented herein.

FIG. 1. Side-view of single layered films of the invention.

FIG. 2. Side-view of various two-layered films of the invention.

FIG. 3. Side-view of various three-layered films of the invention.

### DETAILED DESCRIPTION OF THE INVENTION

As used in the description of the present invention, “effervescent film” is defined as a hot-melt extruded single or multi-layered film that comprises an effervescent couple and an effervescent film binder. As used herein, the term “effervescent granules” refers to granules that consist of an effervescent couple and a suitable hot-melt extrudable effervescent granule binder. These effervescent granules are in some embodiments, prepared by hot-melt extrusion. An “effervescent couple” is defined as a combination of an acidic agent and an alkaline agent that when combined in the presence of water cause the formation of a gas. By way of example, such gases include carbon dioxide, oxygen or chlorine dioxide.

### Effervescent Film

The effervescent film of the invention is generally pliable rather than brittle. It will dissolve/disintegrate at a controlled rate when exposed to a water containing solution. The thickness of the film will be such as to optimize desired behavior, physical characteristics, dissolution rate and rate of effervescence. By “thin film” is meant a film having a thickness generally in the range of about 0.1 mm to about 2.0 mm. The film width can be selected as desired. The effervescent film can be provided in a tape dosage form or in a film short segment dosage form to aid in dispensing and regulating doses.

The effervescent film of the invention will include an effervescent couple or an effervescent granule or a combination thereof. When the film includes an effervescent couple, the alkaline agent and acidic agent which make up the effervescent sample will be dispersed substantially throughout the film. When the film includes an effervescent granule, the film will have localized regions of high concentration of the effervescent couple, which comprises part of the effervescent granule, and the regions will be dispersed within the hot-melt extrudable effervescent film binder which comprises a part of the effervescent film.

When referring to the effervescent film, the term “single or multi-layered” means a film comprising one or more layers. Not all layers need to be effervescent; however, as part of the present invention at least one of the layers will be effervescent. Thus, a multi-layered film-containing dosage form can comprise a plurality of effervescent film layers or an effervescent film in combination with one or more non-effervescent films. A dosage form having a multi-layer configuration is described in the U.S. Pat. No. Re. 33,093, the disclosure of which is hereby incorporated in its entirety by reference.

The effervescent film of the invention is made water soluble or water swellable by including in the film a water soluble or water swellable effervescent film binder. If the binder comprises a combination of water soluble and poorly water soluble components, the combination itself shall be sufficiently water soluble or swellable to provide a rapid release rate of active ingredient from the film.

The term “controlled release film” is taken to mean a film that dissolves, disintegrates or swells in an aqueous solution sufficiently to release all or part of its active ingredient. These preparations may also be created so as to release all or a defined portion of the active ingredient within about 10 minutes after placement of the film in contact with an aqueous solution or a surface having moisture. Thus, the effervescent film has a rapid release rate of active ingredient.

As used herein, the term “hot-melt extrudable” refers to a compound, mixture or formulation that can be hot-melt extruded. A hot-melt extrudable binder is one that is sufficiently rigid at standard ambient temperature and pressure but is capable of deformation or forming a semi-liquid state under elevated heat or pressure. All binders used in the invention are hot-melt extrudable. Both the effervescent film and effervescent granule use hot-melt extrudable binders. Thus, a binder used in the effervescent film is termed the “effervescent film binder”, and a binder used in the effervescent granule is termed the “effervescent granule binder”.

Examples of hot-melt extrudable effervescent film binders include hydroxypropyl cellulose and other hydrophilic cellulosic polymers. In a particular embodiment HPC is the hot-melt extrudable effervescent filing binder. Effervescent film binders can be used in an amount of up to about 99.9 weight percent, and preferably about 40 to about 98 weight percent of the total film composition.

A bioadhesive is defined as a material that adheres to a biological surface such as mucous membrane or skin tissue. A bioadhesive will adherently localize a dosage form onto mucous membrane. The preferred bioadhesive is fibrous or particulate, water swellable but water insoluble. The appropriate ratio of bioadhesive to other film components will provide strong bioadhesion and excellent film integrity. Bioadhesive films as taught in U.S. Pat. No. Re. 33,093 to Schiraldi, the disclosure of which is hereby incorporated in its entirety, can be modified to include an effervescent couple to prepare an effervescent bioadhesive film as contemplated by the indention.

One effervescent bioadhesive film embodiment of the invention comprises polycarbophil (CARBOPOLEX 55® or NOVEON AA-1® from B.F. Goodrich Chemical Co.). The United States Pharmacopeia, 1980 edition, United States Pharmacopeial Convention, Inc., Rockville, Md., page 638, indicates that polycarbophil is a polyacrylic acid cross-linked with divinyl glycol that has a residue on ignition of less than 4.0% and absorbs about 60 times its original weight of water in test B under Absorbing Power (U.S. Pat. No. 4,615,697, Robinson). Other bioadhesive polymers that can be used in this invention include hydrophilic and water-dispersible polymers, having free carboxylic groups and a relatively high base binding capacity. These polymers are polycarboxylated vinyl polymers and polyacrylic acid polymers. Some hydrophilic polysaccharide gums such as guar gum, locust bean gum, psyllium seed gum, and the like are also suitable for use in the formula. The ratio by weight of bioadhesive to active ingredient may be quite broad. In practice, the weight ratio of bioadhesive to active ingredient is about 1:10 to about 10:1.

The bioadhesive effervescent hot-melt extruded films of the invention may require particular hydrophobic or hydrophilic binders in order to obtain suitable product. Suitable hydrophobic binders include cellulose acetate butyrate, cellulose acetate propionate, cellulose propionate high molecular weight (about 200,000), cellulose propionate medium molecular weight (about 75,000), cellulose propionate low molecular weight (about 25,000), cellulose acetate, cellulose nitrate, ethylcellulose, polyvinyl acetate, and the like. Suitable hydrophilic binders include polyvinylpyrrolidone high molecular weight (about 360,000), polyvinylpyrrolidone medium molecular weights (about 24,000 and about 40,000), polyvinyl-pyrrolidone low molecular weight (about 10,000), vinyl alcohol polymer, polyethylene oxide, and the like.

A multi-layered embodiment of the hot-melt extruded effervescent film can comprise a water impermeable layer which can be a cellulose ester based film. The film barrier limits diffusion of the treating agent unidirectionally. Suitable cellulose esters for this purpose are cellulose acetate butyrate, cellulose acetate propionate, cellulose propionate high, medium, and low molecular weights, cellulose acetate, cellulose nitrate, ethylcellulose, polyvinyl acetate, and the like.

As used herein, the term “active ingredient” is defined as a therapeutic compound, a flavoring agent, a sweetening agent, a vitamin, cleansing agent and other such compounds for pharmaceutical, veterinary, horticultural, household, food, culinary, pesticidal, agricultural, cosmetic, herbicidal, industrial, cleansing, confectionery and flavoring applications. The effervescent film can also contain coloring agents, non-effervescent disintegrants, lubricants and the like.

The effervescent film of the invention can be prepared as a single or multi-layer. As a single layer, the effervescent film will be the product of a single extrusion. When a multi-layered effervescent film is involved, the different layer can be coextruded in an extruder equipped with two die slots and then laminated together; alternatively, the different layers can be separately extruded one on the other, and then laminated together. The thickness of the layers in a multi-layered effervescent film may generally be less than the thickness of the individual layer in the single-layer effervescent film.

The film's size and shape can be adapted as desired. The effervescent film should disintegrate substantially upon exposure to water or an aqueous solution. The effervescent granule is present in an amount effective to aid in disintegration of the effervescent film, and preferably when used in a pharmaceutical dosage form, to provide a distinct sensation of effervescence when the film is placed in the mouth of a patient.

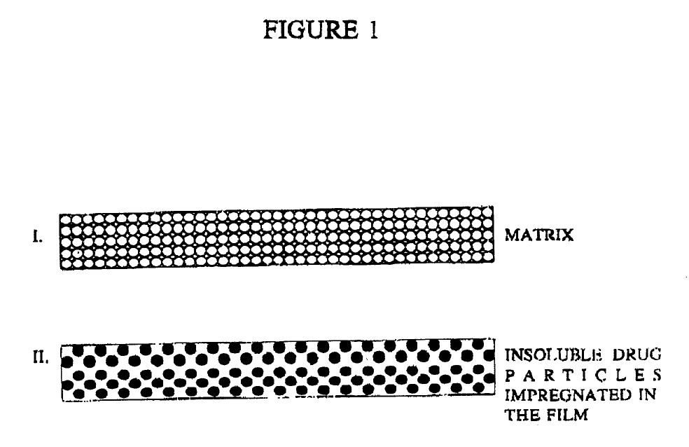

The single layered effervescent bioadhesive films as shown in FIG. 1 ((I) and (II)) contain bioadhesive, effervescent couple, hot-melt extrudable film binders and active ingredient (drug). These compositions may deliver the drug locally to the oral cavity. They are erodible and stay in place for an extended period of time.

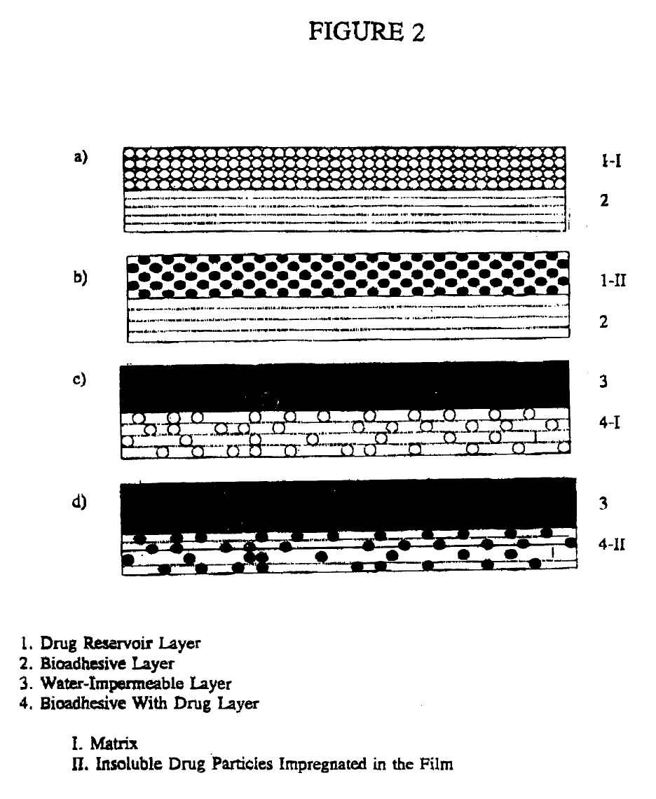

FIGS. 2(a) and (b) depict erodible, two-layered effervescent bioadhesive films which are composed of a water-swellable but water-insoluble bioadhesive layer and an effervescent therapeutic compound reservoir layer. The therapeutic compound is released by diffusion, partition, and/or dissolution from the film which swells or disintegrates. The release of the therapeutic compound and the erosion of the film are controlled by the different proportions of water-swellable hydrogel and hot-melt extrudable film binder. This invention provides local effect to the oral cavity and stays in place for several hours. FIGS. 2(c) and (d) are depictions of erodible or non-erodible two-layered films, which include a bioadhesive effervescent drug containing layer and a water-impermeable layer. The therapeutic compound is released unidirectionally to the buccal tissue and absorbed systemically so as to bypass the first-pass metabolism.

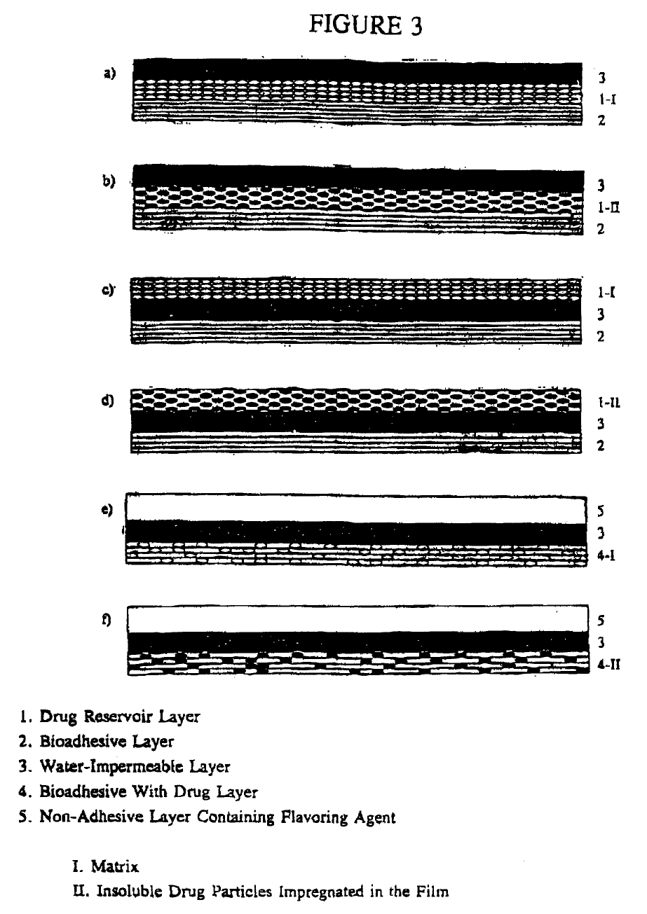

A three-layered bioadhesive effervescent film including a bioadhesive layer, water-impermeable layer, and a flavoring agent is also disclosed in this invention. FIGS. 3(a) and (b) show the erodible and non-erodible, three-layered films containing a bioadhesive layer, a drug reservoir effervescent layer, and a water-impermeable layer. These systems are unidirectional drug diffusion devices. A rate-limiting membrane may be used to replace the drug reservoir layer to accommodate a range of drug properties to generate a once daily delivery-system. FIG. 3(c) and (d) depict other designs of the erodible or non-erodible, three-layered film. The water impermeable layer is in between the bioadhesive layer and drug reservoir layer. The drug is released directly to the oral cavity and provides a local effect. The rate of the drug release is controlled by the ratios of the water-soluble hydrogels and hot-melt extrudable film binder used in the formulation. FIGS. 3(e) and (f) are the erodible or non-erodible, multi-layered devices which are composed of a bioadhesive with drug containing effervescent layer, a water-impermeable layer, and a non-adhesive flavoring layer. The drug releases unidirectionally toward the buccal tissue and is absorbed systemically. Sustained release of the drug from the dosage form is accomplished by controlling the type and the amount of the film binder employed in the bioadhesive-drug layer. The flavoring agent in the non-adhesive layer is released locally to the oral cavity which improves taste of the film, freshens the breath and improves consumer acceptance.

Some embodiments of the invention include a four-layered film comprising a bioadhesive effervescent layer, a drug reservoir effervescent layer, a water-impermeable layer, and a non-adhesive layer. The release of the drug is controlled, at least in part, by the proportion of hot-melt extrudable film binder, effervescent couple and water-insoluble hydrogels in the drug reservoir layer. The drug diffuses across the bioadhesive layer and is absorbed systemically via the buccal tissue. Flavors are added to the non-adhesive layer to provide a palatable taste of the film and improve consumer compliance.

The effervescent sensation is not only pleasant to the patient but also tends to stimulate saliva production, thereby providing additional aqueous medium to aid in further effervescent action. The patient should be able to perceive a distinct sensation of “fizzing” or bubbling as the effervescent film disintegrates in the mouth. The “fizzing” sensation substantially enhances the organoleptic effects of the film. Thus, the amount of effervescent granule present in the effervescent film, to be useful in accordance with the present invention, is also an amount effective to provide a positive organoleptic sensation to a patient. A “positive” organoleptic sensation is one which is pleasant or enjoyable and which can be perceived readily by a normal human being. Thus, once the effervescent film is placed in the patient's mouth, for example, it will disintegrate substantially completely without any voluntary action by the patient. Even if the patient does not chew the film, disintegration will proceed. Upon disintegration of the film, the active ingredient or therapeutic compound, which itself can be particulate, is released and can be swallowed as a slurry or suspension.

When the effervescent film is provided as segments, the film can include surface markings, cuttings, grooves, letters and/or numerals for the purpose of decoration and/or identification. The effervescent film can be provided in a variety of segment shapes: oval, disk, circle, square, rectangle, triangle, parallelogram, diamond, sheet and the like. Such segment shapes are generally prepared by cutting the extruded film into particular shapes. The size of the film segments will be limited by the processing equipment, intended use and product behavior, quality or performance.

Non-effervescent disintegrants include starches such as corn starch, potato starch, pregelatinized and modified starches thereof, cellulosic agents such as Act-di-sol, montmorrilonite clays including cross-linked PVP, sweeteners, bentonite and VEEGUM™, microcrystalline cellulose, alginates, sodium starch glycolate, gums such as agar, guar, locust bean, karaya, pecitin and tragacanth. Disintegrants can comprise up to about 20 weight percent and preferably between about 2 and about 5 percent of the total weight of the composition.

Coloring agents can include titanium dioxide, and dyes suitable for food such as those known as F.D. & C. dyes and natural coloring agents such as grape skin extract, beet red powder, beta-carotene, annato, carmine, turmeric, paprika, etc. The amount of coloring used can range from about 0 to about 2.5 weight percent of the total composition.

Protease inhibitors which can be included in the present film formulations include, by way of example and without limitation, antipain, leupeptin, chymostatin, amistatin and puromycin.

Penetration enhancers which can be included in the present film formulations include, by way of example and without limitation, calcium chelators such as EOTA and polycarboxylic acids; surfactants such as sodium lauryl sulfate, sodium dodecyl sulfate and tween; bile salts such as sodium taurocholate; fatty acids such as oleic and linoleic acid; and non-surfactants such as AZONE and dialkyl sulfoxides.

Flavors incorporated in the composition may be chosen from synthetic flavor oils and flavoring aromatics and/or natural oils, extracts from plants, leaves, flowers, fruits and so forth and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Also useful as flavors are vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors may be present in an amount ranging from about 0.5 to about 3.0 by weight based upon the weight of the composition. Particularly preferred flavors are the grape and cherry flavors and citrus flavors such as orange.

Materials to be incorporated in the effervescent film, other than the effervescent granule, can be pretreated to form granules that readily lend themselves to hot-melt extrusion according to the invention. This process is known as granulation. As commonly defined, “granulation” is any process of size enlargement whereby small particles are gathered together into larger, permanent aggregates to yield a free-flowing composition having a suitable consistency. Such granulated compositions may have consistency similar to that of dry sand. Granulation may be accomplished by agitation in mixing equipment or by compaction, extrusion or agglomeration.

A therapeutic compound, when included in a dosage form including the effervescent film according to the invention, can include at least one psychotropic drug such as a sedative, antidepressant, neuroleptic, or hypnotic. The present invention is especially valuable with psychotropic drugs in that a patient receiving such drugs, particularly a patient in a mental institution, often attempts to hold a conventional pharmaceutical tablet or capsule concealed within his mouth rather than swallow it. The patient may then surreptitiously remove the tablet or capsule when medical personnel are not present. The preferred dosage forms according to this aspect of the present invention are substantially resistant to such concealment, inasmuch as they will disintegrate rapidly even if they are concealed within the mouth.

As the therapeutic compound, use can be made of synthetic antibacterial agents of hardly water-soluble pyridone-carboxylic acid type such as benofloxacin, nalidixic acid, enoxacin, ofloxacin, amifloxacin, flumequine, tosfloxacin, piromidic acid, pipemidic acid, miloxacin, oxolinic acid, cinoxacin, norfloxacin, ciprofloxacin, pefloxacin, lomefloxacin, enrofloxacin, danofloxacin, binfloxacin, sarafloxacin, ibafloxacin, difloxacin and salts thereof. Other therapeutic compounds which can be formulated along with the effervescent granules into the effervescent film include penicillin, tetracycline, erythromycin, cephalosporins and other antibiotics.

Further therapeutic compounds which can be formulated into effervescent films along with the effervescent granules of the invention also include antibacterial substances, antihistamines and decongestants, anti-inflammatories, antiparasitics, antivirals, local anesthetics, antifungal, amoebicidal, or trichomonocidal agents, analgesics, antiarthritics, antiasthmatics, anticoagulants, anticonvulsants, antidepressants, antidiabetics, antineoplastics, antipsychotics, antihypertensives and muscle relaxants. Representative antibacterial substances are beta-lactam antibiotics, tetracyclines, chloramphenicol, neomycin, gramicidin, bacitracin, sulfonamides, aminoglycoside antibiotics, tobramycin, nitrofurazone, nalidixic acid and analogs and the antimicrobial combination of fludalanine/pentizidone. Representative antihistamines and decongestants are perilamine, chlorpheniramine, tetrahydrozoline and antazoline. Representative anti-inflammatory drugs are cortisone, hydrocortisone, betamethasone, dexamethasone, fluocortolone, prednisolone, triamcinolone, indomethacin, sulindac and its salts and corresponding sulfide. A representative antiparasitic compound is ivermectin.

Representative antiviral compounds are acyclovir and interferon. Representative analgesic drugs are diflunisal, aspirin or acetaminophen. Representative antiarthritics are phenylbutazone, indomethacin, silindac, its salts and corresponding sulfide, dexamethasone, ibuprofen, allopurinol, oxyphenbutazone or probenecid. Representative antiasthina drugs are theophylline, ephedrine, beclomethasone dipropionate and epinephrine. Representative anticoagulants are heparin, bishydroxycoumarin, and wararin. Representative anticonvulsants are diphenylhydantoin and diazepam. Representative antidepressants are amitriptyline, chlordiazepoxide perphenazine, protriptyline, imipramine and doxepin. Representative antidiabetics are insulin, somatostatin and its analogs, tolbutamide, tolazamide, acetchexamide and chlorpropamide. Representative antineoplastics are adriamycin, fluorouracil, methotrexate and asparaginase. Representative antipsychotics are prochlorperazine, lithium carbonate, lithium citrate, thioridazine, molindone, fluphenazine, trifluoperazine, perphenazine, amitriptyline and trifluopromazine. Representative antihypertensives are spironolactone, methyldopa, hydralazine, clonidine, chlorothiazide, deserpidine, timolol, propranolol, metoprolol, prazosin hydrochloride and reserpine. Representative muscle relaxants are succinylcholine-chloride, danbrolene, cyclobenzaprine, methocarbamol and diazepam.

The therapeutic compound(s) contained within the effervescent film can be formulated as its pharmaceutically acceptable salts. As used herein, “pharmaceutically acceptable salts” refer to derivatives of the disclosed compounds wherein the therapeutic compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent therapeutic compound which contains a basic or acidic moiety by conventional chemical methods. Generally, such salts can be prepared by reacting the free acid or base forms of these compounds with a predetermined amount of the appropriate base or acid in water or in an organic solvent, or in a mixture of the two. Generally, nonaqueous media are preferred. Lists of suitable salts are found in Remington's Pharmaceutical Sciences, 17th ed., Mack Publishing Company, Easton, Pa., 1985, p. 1418, the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used in this disclosure, the term vitamin refers to trace organic substances that are required in the diet. For the purposes of the present invention, the term vitamin(s) include, without limitation, thiamin, riboflavin, nicotinic acid, pantothenic acid, pyridoxine, biotin, folic acid, vitamin B12, lipoic acid, ascorbic acid, vitamin A, vitamin D, vitamin E and vitamin K. Also included within the term vitamin are the coenzymes thereof. Coenzymes are specific chemical forms of vitamins. Coenzymes include thiamine pyrophosphates (TPP), flavin mononucleotide (FMM), flavin adenine dinucleotive (FAD), nicotinamide adenine dinucleotide (AND), nicotinamide adenine dinucleotide phosphate (NADP), Coenzyme A (CoA), pyridoxal phosphate, biocytin, tetrahydrofolic acid, coenzyme B12, lipoyllysine, 11-cis-retinal, and 1,25-dihydroxycholecalciferol. The term vitamin(s) also includes choline, carnitine, and alpha, beta, and gamma carotenes.

As used in this disclosure, the term “mineral” refers to inorganic substances, metals, and the like required in the human diet. Thus, the term “mineral” as used herein includes, without limitation, calcium, iron, zinc, selenium, copper, iodine, magnesium, phosphorus, chromium and the like, and mixtures thereof.

The term “dietary supplement” as used herein means a substance which has an appreciable nutritional effect when administered in small amounts. Dietary supplements include, without limitation, such ingredients as bee pollen, bran, wheat germ, kelp, cod liver oil, ginseng, and fish oils, amino-acids, proteins and mixtures thereof. As will be appreciated, dietary supplements may incorporate vitamins and minerals.

The amount of therapeutic compound incorporated in each effervescent film segment or tape can be selected according to known principles of pharmacy. An effective amount of therapeutic compound is specifically contemplated. By the term “effective amount”, it is understood that, with respect to, for example, pharmaceuticals, a pharmaceutically effective amount is contemplated. A pharmaceutically effective amount is the amount or quantity of a drug or pharmaceutically active substance which is sufficient to elicit the required or desired therapeutic response, or in other words, the amount which is sufficient to elicit an appreciable biological response when administered to a patient. As used with reference to a vitamin or mineral, the term “effective amount” means an amount at least about 10% of the United States Recommended Daily Allowance (“RDA”) of that particular ingredient for a patient. For example, if an intended ingredient is vitamin C, then an effective amount of vitamin C would include an amount of vitamin C sufficient to provide 10% or more of the RDA. Typically, where the tablet includes a mineral or vitamin, it will incorporate higher amounts, preferably about 100% or more of the applicable RDA.

The therapeutic compound is generally used in finely divided form, i.e. powder or granulate so as to increase the dissolution rate. It is preferable to use a finely powdered therapeutic compound to increase the dissolution rate, more preferably, the therapeutic compound being capable of allowing not less than 80%, desirably not less than 90%, of it to pass through a 100 mesh (150 mu m) screen. The amount of therapeutic compound to be incorporated ranges usually from about 0.1 to 50%, preferably about 1 to 25% by weight based on the effervescent composition, and the ratio may be suitably modified depending on the therapeutic compound then employed. When the therapeutic compound is an acid substance capable of effervescing by reaction with carbonate, the therapeutic compound itself may be used as the acidic agent.

### Effervescent Granule Components

The effervescent granules incorporated in the effervescent film of this invention will comprise an effervescent couple and an effervescent granule binder and can be in the state of a powder or fine particles to increase the dissolution rate, and preferably a particle size such that 90% or more passes an about 16 mesh (1,000μ) screen, and more preferably a particle size such that more than 90% passes an about 18 mesh (850μ) screen. Generally, the larger the effervescent granule, the longer it will take to completely disintegrate. This is particularly true when there are low levels of effervescent couple present in the granules. The effervescent granules will have a controllable rate of effervescence.

As used herein, “effervescence” means the evolution of bubbles of gas from a liquid as the result of a bubble or gas generating chemical reaction. The bubble or gas generating reaction of the effervescent couple in the effervescent granule is most often the result of the reaction of an acidic agent and an alkaline agent. The reaction of these two general classes of compounds produces a gas upon contact with water.

As used herein, the term “acidic agent” refers to any compound or material that can serve as a proton source and can react with the alkaline agent of the invention to form a gas. The acidic agent can have more than one acid dissociation constant, i.e. more than one acid functional group. The acidic agent can be any organic or inorganic acid in the free acid, acid anhydride or acid salt form. An acidic agent which is in solid state at room temperatures and shows pH of about 4.5 or lower when saturated into water at room temperatures or its acid alkali metal salts (e.g. sodium salt, potassium salt, etc.) can be employed. As the acidic agent for the effervescent couple, a compound which is not harmful to animals including humans, is desirably employed. The acidic agent can be tartaric acid, citric acid, maleic acid, fumaric acid, malic acid, adipic acid, succinic acid, lactic acid, glycolic acid, alpha-hydroxy acids, ascorbic acid, amino acids and their alkali hydrogen acid salts. And, even in the case of an acidic agent, such as phosphoric acid or pyrophosphoric acid or other inorganic acids, which is liquid or in liquid state at room temperature or where their acid alkali metal salts are solid at room temperature, those acid alkali metal salts can be employed as acidic agents. Among the above-mentioned acidic agents, those having a relatively large acid dissociation constant about (10³ or more) and a small hygroscopicity (critical humidity at about 30° C. is about 40% RH or more) are preferably employed.

It is preferred if the acidic agent can form a eutectic mixture with an effervescent granule binder. Because these acidic agents are directly ingested, their overall solubility in water is less important than it would be if the effervescent granules of the present invention were intended to be dissolved in a glass of water.

As used herein, the term “alkaline agent” means an alkaline compound that releases a gas, or causes a solution to effervesce, when exposed to a proton source such as an acidic agent or water. The alkaline agent can be a carbon dioxide gas precursor, an oxygen gas precursor or a chlorine dioxide gas precursor.

When the alkaline agent is a carbon dioxide precursor, compounds such as carbonate, sesquicarbonate and hydrogencarbonate salts (in this specification, carbonate and hydrogencarbonate, or bicarbonate, are generically referred to as carbonate) of potassium, lithium, sodium, calcium, ammonium, or L-lysine carbonate, arginine carbonate, sodium glycine carbonate, sodium amino acid carbonate can be used. When the alkaline agent is an oxygen gas precursor, compounds such as anhydrous sodium perborate, effervescent perborate, sodium perborate monohydrate, sodium percarbonate and sodium dichloroisocyannurate can be used. When the alkaline agent is a chlorine dioxide (ClO₂) precursor, compounds such as sodium hypochlorite and calcium hypochlorite can be used. ClO₂ can be used as a chemical sterilizer in cleansing operations.

It is preferred, although not necessary, that both components of the effervescent couple react completely. Therefore, a ratio of components which provides for equal amounts of reaction equivalents is preferred. For example, if the acid used is diprotic, then either twice the amount of a mono-reactive carbonate alkaline agent, or an equal amount of a di-reactive alkaline agent should be used for complete neutralization to be realized. However, in other embodiments of the present invention, the amount of either the acidic agent or the alkaline agent can exceed the amount of the other component. This can be useful to enhance taste and/or performance of a tablet containing an overage of either component.

By controlling the relative ratio of acidic agent: alkaline agent in the effervescent couple, the effervescent granules can be used to regulate the pH of their environment. Thus, the present granules can be used to regulate the pH of body cavities such as the mouth, rectum or vagina.

The ratio of the above-mentioned acidic agent and alkaline agent can also be determined according to the pH required for dissolving an active ingredient included in an effervescent film formulation containing effervescent granules or upon other conditions which a user can contemplate. When the solubility of the active ingredient increases at the acid side, the pH of the solution is lowered by adding the acidic agent in an amount more than equivalent to the alkaline agent. When the solubility of the active ingredient increases at the basic side, the pH of the solution is raised by adding the alkaline agent in an amount more than equivalent to the acidic agent. In either case, the pH near the acidic agent immediately after the dissolution is low, while the pH near an alkaline agent is high. In a case where the solubility of an active ingredient does not depend on pH, the ratio of an acidic agent and an alkaline agent can be optionally selected.

The amount of carbon dioxide precursor, i.e. alkaline agent, to be incorporated in the effervescent granule is proportional to the volume of carbon dioxide gas generated. When it is desired to increase the dissolution rate of an active ingredient included in an effervescent film formulation containing effervescent granules, it can be advantageous to increase the amount of carbon dioxide precursor accordingly, and the amount is usually selected from the range of from about 3% to about 70%, preferably from about 10% to about 70% by weight based on the effervescent granule.

An acidic agent and a carbon dioxide precursor are used respectively in a powdery or granular state, usually 90% or more of them being capable of passing through a 100 mesh (150μ) screen. The particle size of the binder used will usually be about 100 mesh (150μ). In any case, it is generally acceptable that the additional amount of either component can remain unreacted.

Examples of hot-melt extrudable effervescent granule binders include acacia, tragacanth, gelatin, starch, cellulose materials such as methyl cellulose and sodium carboxymethyl cellulose, alginic acids and salts thereof, polyethylene glycol, guar gum, polysaccharide, sugars, invert sugars, poloxomers (PLURONIC F68, PLURONIC F127), collagen, albumin, gelatin, cellulosics in nonaqueous solvents, and combinations of the above and the like. Other binders include, for example, polypropylene glycol, polyoxyethylene-polypropylene copolymer, polyethylene ester, polyethylene sorbitan ester, polyethylene oxide or combinations thereof and the like. Hydrophobic binders can also be used in the invention.

Effervescent granule binders can be used in an amount of up to about 60 weight percent and preferably less than about 10 weight percent and more preferably about 3 to about 8 weight percent of the total effervescent granule composition. While the melting and/or softening point temperatures of these binders usually rise with increase of their molecular weights, preferable ones are those with a melting or softening point temperature less than about 150° C. However, binders having melting or softening points greater than about 150° C. can be used. Hot-melt extrudable binders having a melting or softening point temperature greater than about 150° C. will require use of a plasticizer during hot-melt extrusion such that the binder melting or softening point temperature will be lowered below 150° C. Among the above-mentioned binders, polyethylene glycol is preferable, and that having a molecular weight of about 1000 to 8000 Da is more preferable. The binder can be used in any form such as powder, granules, flakes or heat-molten liquid.

By “controllable rate of effervescence” is meant that the rate of effervescence can be controlled such that a defined rapid rate of effervescence by an effervescent granule is achieved. The rate of effervescence by an effervescent granule is controlled as detailed below.

When referring to the rate of effervescence as “rapid”, it is understood that the effervescent granules of the present invention should disintegrate in an aqueous solution in less than 10 minutes, and desirably between about 15 seconds and about 7 minutes. In a particularly preferred embodiment according to the present invention, the effervescent granules should dissolve in an aqueous solution in between about 8 seconds and about 5 minutes. Disintegration time can be approximated by observing the disintegration time of the effervescent granules immersed in water at about 37° C. The disintegration time is the time from immersion to substantially complete disintegration of the effervescent granules as determined by visual observation. As used in this disclosure the term “complete disintegration” of the effervescent granules refers to the dissolution or disintegration of the effervescent granules. Disintegration times referred to in this disclosure should be understood as determined by the method used herein unless otherwise specified.

Control of the rate of effervescence can be achieved by varying the relative amounts of the components in the effervescent granule. Thus, by increasing the amount of hot-melt extrudable binder relative to the total weight of the effervescent granule, a less friable and stronger granule can be generally prepared. Conversely, by decreasing the amount of hot-melt extrudable binder relative to the total weight of the effervescent granule, a more friable or weaker granule can be generally prepared. Hydrophobic binders will generally tend to have a greater impact upon granule hardness than hydrophilic binders.

Generally, forming a eutectic mixture between the acidic agent and the hot-melt extrudable effervescent granule binder before hot-melting extruding with the alkaline agent will yield effervescent granules that are harder and thus slower dissolving than those prepared by hot-melt extruding the binder, acidic agent and alkaline agent components together simultaneously.

Having an excess of either the acidic agent or alkaline agent in the effervescent granule will generally result in increased rate of effervescence when compared to an effervescent granule having the same amounts, on an equivalent basis, of both agents. Regardless of whether either agent is in excess, the total amount of gas produced by an effervescent granule will not exceed the theoretical amount of gas that should be produced by the agent serving as the limiting reagent.

Including a plasticizer in the present effervescent granules may be used in some embodiments of the invention to alter its rate of effervescence. Generally, increasing the amount of plasticizer present will increase or prolong the time of effervescence.

Generally, the more hydrophobic the effervescent granule binder, the slower the rate of effervescence. The solubility and rate of dissolution of a hydrophobic binder are important factors to consider as the level of binder in the effervescent granule is increased. The rate of effervescence can also be controlled by varying the hydrophilicity or hydrophobicity of the hot-melt extrudable effervescent granule binder. For example, one can prepare an effervescent granule having a rapid rate of effervescence by a water soluble hot-melt extrudable binder such as an electrolyte or nonelectrolyte such as xylitol, which is hydrophilic and can form a eutectic mixture with an appropriate acidic agent during hot-melt extrusion.

The effervescent granule can also employ a surface active agent or cosolvent that improves wetting or disintegration of the effervescent granule.

Thus, rate of effervescence of the effervescent granule can be controlled by: (1) varying the relative amounts of the components; (2) optionally forming a eutectic mixture between the acidic agent and hot-melt extrudable effervescent granule binder; (3) varying acidic agent: alkaline agent ratio; (4) hydrophilicity vs. hydrophobicity of hot-melt extrudable effervescent granule binder; (5) varying the effervescent couple: hot-melt extrudable effervescent granule binder ratio; and (6) varying the amount of plasticizer present.

The amount of effervescent granules of the present invention useful for the formation of effervescent films, in general, should range from about 0 to about 25% by weight of the final film composition, and preferably between about 3 and about 10% by weight thereof.

### Hot-Melt Extrusion

In one aspect of this invention, the effervescent film is produced by a hot-melt extrusion method. In some embodiments, an effervescent couple, an active ingredient, a hot-melt extrudable water soluble or swellable effervescent film binder and optionally other components, such as a plasticizer, are placed into a mixer or hopper and mixed until thoroughly blended to form an effervescent mixture. The effervescent mixture is then hot-melt extruded at a rate and temperature sufficient to melt or soften the effervescent film binder, to minimize degradation of film components and to form a film extrudant which is subsequently rolled into a tape or segmented by chopping or cutting. The film made by this process will have its components essentially thoroughly dispersed throughout the film.

In another aspect of this invention, the effervescent film is produced by mixing an effervescent granule, an active ingredient, a hot-melt extrudable water soluble or swellable effervescent film binder and optionally other components in a hopper or mixer until thoroughly blended and then hot-melt extruding the mixture at a rate and temperature sufficient to melt or soften the effervescent film binder, to minimize degradation of film components and to form a film extrudant which is subsequently rolled into a tape or segmented by chopping or cutting. The film made by this process will have localized regions containing high concentrations of effervescent couple dispersed within regions containing the active ingredient, effervescent film binder and other optional components.

When the effervescent film of the invention contains effervescent granules, such granules can be prepared as follows. An acidic agent and an alkaline agent, preferably a carbon dioxide precursor, and a hot-melt extrudable effervescent granule binder are placed into a mixer or hopper and agitated (blended) until thoroughly mixed to form an effervescent mixture. The effervescent granule components can be solid or liquid prior to hot-melt extrusion. The effervescent mixture is then hot-melt extruded at a rate and temperature sufficient to melt or soften the binder, to minimize degradation of the components and to form an extrudant which is subsequently ground or chopped into effervescent granules.

In another aspect of the invention, the effervescent granule used in an effervescent film is produced by a hot-melt extrusion process as follows. An acidic agent and a hot-melt extrudable effervescent granule binder, capable of forming a eutectic mixture with the acidic agent, are placed into a mixer and agitated until thoroughly mixed to form a mixture which is hot-melt extruded and ground to form a granular eutectic mixture. An alkaline agent, such as a carbon dioxide precursor, is added to the granular eutectic mixture and thoroughly blended to form an effervescent mixture. The effervescent mixture is then hot-melt extruded at a rate and temperature sufficient to melt or soften the eutectic mixture, to minimize degradation of the components, e.g. degradation of NaHCO₃ to Na₂CO₃, and to form an extrudant which is subsequently ground or chopped into effervescent granules.

As used herein, the term “effervescent mixture” means a granular or particulate mixture comprising an acidic agent, an alkaline agent and a hot-melt extrudable binder which when placed in water will cause effervescence. As used herein, the term “eutectic mixture” means a mixture of an acidic agent and a hot-melt extrudable effervescent granule binder that has been hot-melt extruded and that melts or softens at a temperature lower than the melting or softening temperature of the hot-melt extrudable effervescent granule binder neat. The eutectic mixture can be a full or partial mixture and can be referred to as a “solid solution.”

Many conditions can be varied during the extrusion process to arrive at a particularly advantageous formulation. Such conditions include, by way of example, formulation composition, feed rate, operating temperature, extruder screw RPM, residence time, die configuration, heating zone length and extruder torque and/or pressure. Methods for the optimization of such conditions are known to the skilled artisan.

The rate at which the hot-melt extrusion is conducted can also vary widely. The rate will be such that degradation of the components of the mixture being extruded will be minimized. Such rate can be easily determined experimentally and will vary according to the particular mixture being extruded. Generally, the extrusion rate is such that the time of exposure of the components to the elevated temperature is less than 5 minutes and preferably less than 2 minutes.

The hot-melt extrusion process preferably employed is conducted at an elevated temperature, i.e. the heating zone(s) of the extruder is above room temperature (about 20° C.). It is important to select an operating temperature range that will minimize the degradation or decomposition of the effervescent composition during processing. The operating temperature range is generally in the range of from about 50° C. to about 150° C. as determined by the setting for the extruder heating zone(s). The temperature of the mixture being hot-melt extruded will not exceed 150° C. and preferably will not exceed 120° C. The hot-melt extrusion is conducted employing a dry granular or powdered feed.

The extruder used to practice the invention can be any such commercially available model equipped to handle dry feed and having a solid conveying zone, one or multiple heating zones, and an extrusion die. A two stage single screw extruder, such as that manufactured by BRABENDER or KILLION are two such apparati. It is particularly advantageous for the extruder to possess multiple separate temperature controllable heating zones.

When preparing the effervescent film, the extruder will be equipped with a film die which has an adjustable height and width to prepare effervescent films of varying thicknesses and widths, respectively. When preparing a multi-layered film, the extruder can be equipped with a sheet die having more than one die slot. When preparing effervescent granules, which can be included in the effervescent film, the extruder will be equipped with a die having a spherical orifice which is available in various diameters to prepare effervescent granules of varying diameters.

The extruder is generally used along with a chopper or grinder to provide segments of effervescent film or effervescent granules. By varying extrusion rate and chopper speed, the length of effervescent film segments and size of effervescent granules can be controlled and optimized for particular applications and embodiments of the invention. Any film remaining after the segments have been collected can be recycled by milling to the desired particle size and mixing with virgin material.

Extruder configuration will generally need to be varied according to whether a single or multi-layered effervescent film containing formulation is being prepared. Single-layer effervescent films can suitably be prepared as described above and in Example 3. Multi-layered effervescent films can be prepared as described by Schiraldi et al. (U.S. Pat. No. Re. 33,093) the disclosure of which is hereby incorporated by reference in its entirety or as described in Example 4.

When higher melting temperature, higher molecular weight or high softening temperature binders are employed, the hot-melt extrusion may require higher processing temperature, pressure and/or torque than when binders having a lower molecular weight, melting or softening temperature are employed. By including a plasticizer, and, optionally, an antioxidant, in a formulation, processing temperature, pressure and/or torque may be reduced. Plasticizers are not required in order to practice the invention but can be included when desired to tailor film flexibility and to aid in cutting and processing the film. Their addition to the formulation is contemplated as being within the scope of the invention. Plasticizers are advantageously included in the effervescent granule or effervescent film when hot-melt extrudable binders having a melting or softening point temperature greater than 150° C. are employed.

As used herein, the term “plasticizer” includes all compounds capable of plasticizing a hot-melt extrudable binder used in invention. The plasticizer should be able to lower the melting temperature or glass transition temperature (softening point temperature) of the hot-melt extrudable binder. Plasticizers, such as low molecular weight PEG, generally broaden the average molecular weight of the hot-melt extrudable binder thereby lowering its glass transition temperature or softening point. Plasticizers also generally reduce the viscosity of a polymer melt thereby allowing for lower processing temperature and extruder torque during hot-melt extrusion. It is possible the plasticizer will impart some particularly advantageous physical properties to the effervescent granule and effervescent film of the invention.

Plasticizers useful in the invention can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin.

Such plasticizers can also be ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate. All such plasticizers are commercially available from sources such as Aldrich or Sigma Chemical Co.

It is contemplated and within the scope of the invention, that a combination of plasticizers may be used in the present formulation. One advantageous combination is that comprised of poly(ethylene glycol) and low molecular weight poly(ethylene oxide). The PEG based plasticizers are available commercially or may be made by a variety of methods, such as disclosed in Poly(ethylene glycol) Chemistry: Biotechnical and Biomedical Applications (J. M. Harris, Ed.; Plenum Press, NY) the teachings of which are hereby incorporated by reference.

The amount of plasticizer used in the effervescent granule or effervescent film will depend upon its composition, physical properties, effect upon the effervescent granule or effervescent film, interaction with other components of the granule or film and other such reasons. Generally, the plasticizer content will not exceed about 40% wt. of the effervescent granules and about 50% wt. of the effervescent film.

Some embodiments of the present invention provide an effervescent water soluble or swellable rapid release hot-melt extruded single layered thin film comprising: a water soluble or swellable hot-melt extrudable effervescent film binder present in an amount of about 40% to about 99.9% by weight; a plasticizer present in an amount of about 0% to about 40% by weight; and an effervescent granule present in an amount of about 3% to about 15% by weight;

said weight percentages being based upon the final weight of said effervescent film.

The foregoing will be better understood with reference to the following examples which detail certain procedures for the manufacture of films according to the present invention. All references made to these examples are for the purposes of illustration. They are not to be considered limiting as to the scope and nature of the present invention.

### EXAMPLE 1

### PREPARATION OF EFFERVESCENT GRANULES

The following general procedure can be used to prepare a variety of effervescent granules used in the effervescent film according to the present invention.

All materials to be used are passed through a fine screen (100 mesh). The materials are then dried at 40° C. for 24 hours, preferably in a vacuum. The following steps are conducted in an atmosphere having a low relative humidity. All materials are then mixed in a twin shell blender for 5-10 minutes until a uniform blend is achieved. Then, using a hot-melt extrusion apparatus, the powder blend is subjected to a temperature of less than or equal to about 120° C. at a rate and for a period of time sufficient to melt or soften the binder to form agglomerates of the effervescent couple in an extrudant which is either chopped or ground. The extruded granules are then screened and stored at a low relative humidity for subsequent incorporation into a variety of pharmaceutical dosage forms.

The following materials can be used to prepare the effervescent granules according to the procedure just described.

| | Ingredients | Amount (% Wt.) |
|---|---|---|
| A. | | |
| | NaHCO₃ | 52 |
| | Citric Acid | 14 |
| | Tartaric Acid | 28 |
| | PEG 1,000 | 6 |
| B. | | |
| | NaHCO₃ | 55 |
| | Citric Acid | 13.5 |
| | Tartaric Acid | 24 |
| | PEG 4,000 | 7.5 |
| C. | | |
| | Sodium Glycine Carbonate | 58 |
| | Citric Acid | 15 |
| | Tartaric Acid | 21 |
| | Pluronic F68 | 6 |
| D. | | |
| | NaHCO₃ | 54 |
| | Citric Acid | 16 |
| | Tartaric Acid | 24 |
| | PEG 20,000 | 3 |
| | PEG 400 | 3 |
| E. | | |
| | NaHCO₃ | 50 |
| | Citric Acid | 14 |
| | Tartaric Acid | 28 |
| | PEG 8,000 | 8 |
| F. | | |
| | KHCO₃ | 62 |
| | Fumaric Acid | 5 |
| | Citric Acid | 8 |
| | Tartaric Acid | 18 |
| | PEG 6,000 | 7 |
| G. | | |
| | NaHCO₃ | 55 |
| | NaH₂PO₄ | 37.5 |
| | Pluronic F127 | 7.5 |
| H. | | |
| | NaHCO₃ | 54 |
| | Fumaric Acid | 3 |
| | Maleic Acid | 5 |
| | Citric Acid | 13 |
| | Tartaric Acid | 18 |
| | PEG 1,000 | 3 |
| | Pluronic F68 | 4 |
| I. | | |
| | NaHCO₃ | 56 |
| | Citric Acid | 37 |
| | Cetyl alcohol | 2 |
| | Stearyl alcohol | 5 |
| J. | | |
| | NaHCO₃ | 51 |
| | Citric Acid | 34 |
| | Xylitol | 15 |
| K. | | |
| | NaHCO₃ | 50 |
| | Citric Acid | 40 |
| | Xylitol | 10 |

In this example, xylitol and citric acid are first hot-melt extruded to form a eutectic mixture which is then hot-melt extruded with NaHCO₃ to form the effervescent granule.

### EXAMPLE 2

### DETERMINATION OF EFFERVESCENT GRANULE DISSOLUTION RATE

This is a visual end-point test for determining effervescent granule solubility.

Effervescent granule (2.0 grams) was added rapidly in one portion to a very gently stirred (less than 60 rpm) beaker containing water (1.0 L) at about 20°-25° C. The endpoint was visually determined by observing cessation of effervescence or complete dissolution of effervescent granules.

### EXAMPLE 3

### PREPARATION OF A SINGLE LAYERED EFFERVESCENT FILM BY HOT MELT EXTRUSION

The effervescent couple (EC) used to make the effervescent film can be in the form of an effervescent granule (EG) or a mixture of acidic agent and alkaline agent powders. The effervescent film is generally made as follows:

HPC (750 g) is mixed with plasticizer (50 g) by directly adding the plasticizer to or spraying the plasticizer onto the HPC. Following a 10 min. mixing period, effervescent granule (50 g) and active ingredient (150 g) are added to the HPC/plasticizer mixture and blended. The resulting mixture is hot-melt extruded into a film using a KILLION or JOHNSON extruder equipped with a single three stage screw at temperatures ranging from about 50° to about 180° C. The extruded film is cut into segments with a chopper.

The following exemplary ingredients can be used in preparing effervescent films according to the invention.

| | | Amount (% Wt.) |
|---|---|---|
| A. | Ingredients | |
| | HPC | 70 |
| | triethylcitrate (TEC) | 3 |
| | poly(ethylene glycol) | |
| | 600 (PEG) | 2 |
| | PEG 4000 | 3 |
| | EG | 7 |
| | APAP (acetaminophen) | 15 |
| B. | Ingredients | |
| | HPC | 77 |
| | EG (effervescent couple) | 9 |
| | PEG 1000 | 5 |
| | PEG 400 | 3 |
| | Hydroxypropyl | |
| | methylcellulose (HPMC) | 5 |
| | CPM (chlorpheniramine | 1 |
| | maleate) | |
| C. | Ingredients | |
| | HPC | 65 |
| | EC (effervescent couple) | 7 |
| | PEG 4000 | 5 |
| | PEG 600 | 3 |
| | Carbomer | 2 |
| | Hydroxyethylcellulose | |
| | (HEC) | 3 |
| | APAP | 15 |
| D. | Ingredients (Bioadhesive) | |
| | HPC | 80 |
| | EC | 5 |
| | PEG 1000 | 2 |
| | PEG 400 | 3 |
| | Polyacrylic acid | 3 |
| | Poloxomer | 2 |
| | pseudoephedrine HCl | 5 |

### EXAMPLE 4

### PREPARATION OF AN EFFERVESCENT BIOADHESIVE MULTI-LAYERED EFFERVESCENT FILM BY HOT-MELT EXTRUSION

A multi-layered effervescent film of the invention can be prepared by following the procedure of Example 3 and modifying it as follows.

A single-layered effervescent film is prepared according to Example 3A. The ingredients for Example 3D are placed in an extruder hopper and extruded. After this film exits the extruder, it is pressed onto the effervescent film in a continuous feed fashion. Thus, the final bi-layered film has one effervescent layer and one bioadhesive layer.

The above is a detailed description of particular embodiments of the invention. Those with skill in the art should, in light of the present disclosure, appreciate that obvious modifications of the embodiments disclosed herein can be made without departing from the spirit and scope of the invention. All of the embodiments disclosed and claimed herein can be made and executed without undue experimentation in light of the present disclosure. The full scope of the invention is set out in the claims that follow and their equivalents. Accordingly, the claims and specification should not be construed to unduly narrow the full scope of protection to which the present invention is entitled.

### REFERENCES

The following references, to the extent they provide exemplary procedural or other details supplementary to those set forth herein, are specifically incorporated herein by reference.

| 1. | U.S. Pat. No. 1,262,888 | (4/1918) to Westlake |
|---|---|---|
| 2. | U.S. Pat. No. 3,962,417 | (6/1976) to Howell |
| 3. | U.S. Pat. No. 4,613,497 | (9/1986) to Chaukin |
| 4. | U.S. Pat. No. 4,639,368 | (1/1987) to Niazi |
| 5. | U.S. Pat. No. 4,687,662 | (8/1987) to Schobel |
| 6. | U.S. Pat. No. 4,725,427 | (2/1988) to Ashmead |
| 7. | U.S. Pat. No. 4,753,792 | (6/1988) to Aberg |
| 8. | U.S. Pat. No. 4,940,588 | (7/1990 to Sparks |
| 9. | U.S. Pat. No. 5,055,306 | (2/1991) to Barry |
| 10. | U.S. Pat. No. 3,667,929 | (6/1972) to Fleming |
| 11. | U.S. Pat. No. 4,153,678 | (5/1979) to Quinlan |
| 12. | U.S. Pat. No. 4,267,164 | (5/1981) to Yeh et al. |
| 13. | U.S. Pat. No. 5,100,674 | (3/1992) to Ser et al. |
| 14. | Pharmaceutical Dosage Forms-Tablets vol. 1, 2nd edition, | |
| | Herbert A. Lieberman, ed. pp. 372-376. | |
| 15. | BE664197 | (11/1965) |
| 16. | EP0189114 | (7/1986) |
| 17. | EP0190689 | (8/1986) |
| 18. | EP0217631 | (4/1987) |
| 19. | DE1938709 | (4/1970) |
| 20. | DE2020893 | (11/1970) |
| 21. | DE2213604 | (6/1973) |
| 22. | GB917456 | (2/1963) |
| 23. | GB1055854 | (1/1967) |
| 24. | GB1276839 | (6/1972) |
| 25. | GB1300998 | (12/1972) |
| 26. | GB1370766 | (10/1974) |
| 27. | GB2019844 | (11/1979) |
| 28. | GB2083997 | (4/1982) |
| 29. | EP0396335 | (11/1990) |
| 30. | GB0003160 | (10/1872) |

What is claimed is:

1. An effervescent water soluble or swellable controlled release hot-melt extruded thin film comprising:
a water soluble or swellable hot-melt extrudable effervescent film binder present in an amount of about 40% to about 99.9% by weight of the film;
an effective amount of an active ingredient present in an amount of about 0.05% to about 60% by weight of the film;
a plasticizer present in an amount of about 0% to about 50% of the film; and
an effervescent couple present in an amount of about 0.1% to about 60% of the film.

2. A method for preparing an effervescent controlled release hot-melt extruded thin film comprising:
providing a hot-melt extruded effervescent granule comprising: a hot-melt extrudable effervescent granule binder and an effervescent couple;
mixing said effervescent granule with a hot-melt extrudable effervescent film binder, and an active ingredient to form an effervescent mixture; and
hot-melt extruding said effervescent mixture to form said film.

3. The method of claim 2 wherein the effervescent granule is mixed with a plasticizer.

4. An effervescent controlled release hot-melt extruded thin film comprising:
a water soluble or swellable hot-melt extrudable effervescent film binder present in an amount of about 40% to about 99.9% by weight of the film;
a plasticizer present in an amount of about 0% to about 40% by weight of the film; and
an effervescent granule present in an amount of about 3% to about 15% by weight of the film.

5. The effervescent water soluble or swellable controlled release hot-melt extruded thin film of claim 4 wherein the hot-melt extruded thin film is a single layer film.

6. An effervescent controlled release hot-melt film of claim 1 or 3, further comprising a bioadhesive.

7. The effervescent controlled release hot-melt extruded thin film of claim 1 or 3 further defined comprising a plasticizer in an amount of about 0.5% to about 60% by weight of the film.

8. The effervescent controlled release hot-melt extruded thin film of claim 1 or 3 further defined as a multi-layer film.

9. A method for preparing a bioadhesive effervescent controlled release hot-melt extruded soluble or swellable two-layered thin film comprising:
providing a bioadhesive thin film;
mixing an effervescent couple with a hot-melt extrudable water-soluble or swellable effervescent film binder, an effective amount of an active ingredient to form an effervescent mixture;
hot-melt extruding said effervescent mixture to form an effervescent single-layered hot-melt extruded water soluble or swellable film; and
pressing said effervescent single-layered hot-melt extruded water soluble or swellable film onto said bioadhesive thin film.

10. The method of claim 9 wherein the effervescent couple is mixed with a plasticizer.
